(19) United States
(12) Patent Application Publication
(10) Pub. No.: US 2011/0184525 A1
(43) Pub. Date: Jul. 28, 2011

(54) Knee joint endoprosthesis

(75) Inventor: Thomas Hagen, Tuttlingen (DE)

(73) Assignee: AESCULAP AG, Tuttlingen (DE)

(21) Appl. No.: 12/984,933

(22) Filed: Jan. 5, 2011

(30) Foreign Application Priority Data
Jan. 13, 2010 (DE) 10 2010 000 067.1

(51) Int. Cl. A61F 2/38

(52) U.S. Cl. 623/20.29

(57) ABSTRACT

A knee joint endoprosthesis comprises a femoral component, a tibial component and a meniscal component mounted between the femoral component and the tibial component. The meniscal component is mounted on the tibial component for rotation about an axis of rotation extending at the medial side, a rotation guiding device being provided to force rotational movement of the meniscal component relative to the tibial component about the axis of rotation as a result of pivotal movement of the femoral component and the tibial component relative to each other about a pivot axis extending transversely to the axis of rotation. The rotation guiding device comprises interacting first and second guiding elements arranged or formed, on the one hand, on the femoral component and, on the other hand, on the tibial component.

### CROSS-REFERENCE TO RELATED APPLICATIONS

The present disclosure relates to the subject matter disclosed in German application number 10 2010 000 067.1 of Jan. 13, 2010, which is incorporated herein by reference in its entirety and for all purposes.

### FIELD OF THE INVENTION

The invention relates to knee joint endoprostheses, in general, and quite specifically, to a knee joint endoprosthesis with a femoral component, a tibial component and a meniscal component mounted between the femoral component and the tibial component.

### BACKGROUND OF THE INVENTION

Knee joint endoprostheses of the kind described at the outset are used, in particular, when natural knee joints suffer damage owing to traumas or long-term wear, which permanently impairs the quality of life of those affected. Known knee joint endoprostheses have various shortcomings, and so the present invention is concerned with improving knee joint endoprostheses of the kind described at the outset, in particular, with respect to their functionality.

### SUMMARY OF THE INVENTION

In a first aspect of the invention, a knee joint endoprosthesis comprises a femoral component, a tibial component and a meniscal component mounted between the femoral component and the tibial component. The meniscal component is mounted on the tibial component for rotation about an axis of rotation extending at the medial side, and a rotation guiding device is provided to force rotational movement of the meniscal component relative to the tibial component about the axis of rotation as a result of pivotal movement of the femoral component and the tibial component relative to each other about a pivot axis extending transversely to the axis of rotation. The rotation guiding device has interacting first and second guiding elements arranged or formed, on the one hand, on the femoral component and, on the other hand, on the tibial component.

### BRIEF DESCRIPTION OF THE DRAWING FIGURES

The foregoing summary and the following description may be better understood in conjunction with the drawing figures, of which:

FIG. 1 shows an anterior side view of an embodiment of a knee joint endoprosthesis;

FIG. 2 shows a perspective view of the embodiment from FIG. 1 in an extended position;

FIG. 3 shows an exploded representation of the embodiment from FIG. 2,

FIG. 4 shows a view of the embodiment in analogy with FIG. 2 in a bent position; and

FIG. 5 shows a plan view of the embodiment in the bent position shown in FIG. 4.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The invention relates to a knee joint endoprosthesis with a femoral component, a tibial component and a meniscal component mounted between the femoral component and the tibial component, the meniscal component being mounted on the tibial component for rotation about an axis of rotation extending at the medial side, wherein a rotation guiding device is provided to force rotational movement of the meniscal component relative to the tibial component about the axis of rotation as a result of pivotal movement of the femoral component and the tibial component relative to each other about a pivot axis extending transversely to the axis of rotation, the rotation guiding device having interacting first and second guiding elements arranged or formed, on the one hand, on the femoral component and, on the other hand, on the tibial component.

Such a knee joint endoprosthesis enables eccentric rotation of the meniscal component relative to the tibial component, but nevertheless an improved stabilization is achieved overall by the rotation guiding device provided. The guidance is achieved, in particular, by the interacting first and second guiding elements which are arranged, on the one hand, on the femoral component and, on the other hand, on the tibial component. Optionally, further guiding elements may, of course, also be provided, but it is also possible to provide two guiding elements only in the manner indicated. The proposed knee joint endoprosthesis makes it possible, as a result of bending of the knee, to simultaneously force rotation of the meniscal component relative to the tibial component about the axis of rotation. In this way, physiological knee kinematics can be approximately reproduced with the proposed knee joint endoprosthesis. The rotation guiding device is, in particular, suited to simulate the function of missing cruciate ligaments, whereby the femoral component can be moved in the posterior direction on the meniscal component during flexion and the joint can also be stabilized in flexion. All in all, strength of the leg mechanics is thereby increased and improved quadriceps power is achieved. Depending on the configuration, in particular, also guidance of the patella can be improved. Patellar complications resulting from implantation of a knee joint endoprosthesis can be reduced in this way. Furthermore, depending on its design, a greater bending capability of the knee joint endoprosthesis can also be achieved.

It is expedient for the femoral component to comprise a medial condyle and a lateral condyle, which have a medial condylar surface and a lateral condylar surface, and for the meniscal component to comprise a medial joint surface and a lateral joint surface on which the medial and lateral condylar surfaces bear at least partially. Owing to the construction of the described condyles and joint surfaces, sliding and/or rolling movement, depending on the configuration of the shape of the condyles and joint surfaces, can be achieved between the femoral component and the meniscal component.

Particularly expedient guidance of the femoral component on the meniscal component can, for example, be achieved by the medial and/or the lateral condylar surface comprising a concavely curved condylar surface region, and by the medial and/or the lateral joint surface comprising a convexly curved joint surface region corresponding to the medial and/or lateral condylar surface.

In accordance with a further embodiment of the invention, it may be provided that radii of curvature of the medial and/or lateral joint surfaces are larger than radii of curvature of the medial and/or lateral condylar surfaces. In this way, it is possible to also superimpose rolling movement on sliding movement between the components of the prosthesis. In particular, as a result of bending of the knee, the femoral component can thus be moved relative to the meniscal component in the posterior direction, as is the case with a natural knee joint.

It is of advantage for the axis of rotation to be defined by a rotary bearing formed between the tibial component and the meniscal component. A particularly simple and compact construction of the knee joint endoprosthesis is thereby achieved.

The rotary bearing is of particularly simple construction if it comprises interacting first and second bearing elements which are arranged or formed, on the one hand, on the tibial component and, on the other hand, on the meniscal component. The bearing elements may be releasably connectable or permanently connected to the tibial component and/or the meniscal component. In particular, they may also be formed integrally with the respective components of the knee joint endoprosthesis.

A rotary bearing enables particularly simple and safe guidance if the first and second bearing elements take the form of a projection and a corresponding recess. The projection may be selectively formed on the tibial component or on the meniscal component. In a corresponding manner, the recess may be formed on the respective other component. The recess may take the form of a depression or also an opening on one of the two components of the knee joint endoprosthesis.

The axis of rotation may also be defined in a simple way by the first and second bearing elements being formed rotationally symmetrically in relation to the axis of rotation.

In order to achieve a defined rotational movement about the axis of rotation, it is expedient for the first and second bearing elements to comprise interacting first and second guiding surfaces which are formed rotationally symmetrically in relation to the axis of rotation. In this way, it is, in particular, possible to prevent movement of the meniscal component and the tibial component relative to each other in a direction transverse to the axis of rotation when the first and second bearing elements interact with each other, i.e., in particular, are in engagement with each other.

Preferably, one of the bearing elements is of cylindrical shape and the other bearing element is of hollow-cylindrical shape. In particular, they can thus be inserted in each other with positive locking or partially with positive locking. It is also conceivable to form the cylindrically shaped bearing element cylindrically only in sections thereof, i.e., to provide recesses on the bearing element, which extend in the radial direction in relation to the cylindrical guiding surface. Such a configuration does not restrict the functioning in interaction with a corresponding hollow-cylindrically shaped bearing element. In order to limit an angle of rotation, the bearing elements may also be cylindrically or hollow-cylindrically shaped over only a certain angular range, in order to thus form rotation stops. In this case, an angular range predefined by the bearing element which is hollow-cylindrically shaped in sections is preferably greater than that of the bearing element which is cylindrically shaped only partially.

Expediently, the first and second guiding surfaces are formed coaxially in relation to the axis of rotation. Two or more guiding surfaces may also be selectively provided on the respective guiding element with different radii of curvature or diameters, in order to also simultaneously define, in addition to a limitation of movement in a direction transverse to the axis of rotation, axial stops in a direction parallel to the axis of rotation.

In accordance with a further embodiment, it may be provided that the rotary bearing takes the form of a ball joint bearing. A ball joint bearing defines, in principle, a plurality of axes of rotation. In particular, the limitation to a corresponding axis of rotation may be achieved by movement of the meniscal component and the tibial component relative to each other being limited in one or to only one plane, i.e., a solely two-dimensional movement of tibial component and meniscal component relative to each other is enabled.

A ball joint bearing can be particularly simply constructed if it comprises first and second ball joint surfaces, if one of the ball joint surfaces is of hollow-spherical shape and the other ball joint surface is of spherical shape, and if one of the bearing elements comprises one ball joint surface and the other bearing element comprises the other ball joint surface. The first and second ball joint surfaces may be selectively formed on the meniscal component or on the tibial component. No restriction, for example, is required that the hollow-spherical joint surface has to be provided on the meniscal component. In particular, it could also be formed or provided on the tibial component.

To ensure safe and defined guidance of meniscal component and femoral component relative to each other about an axis of rotation defined by the rotary bearing, it is expedient for the first and second ball joint surfaces to have identical or substantially identical radii of curvature.

Preferably, the rotation guiding device is configured to force rolling movement of the femoral component and the meniscal component on each other. In this way, the rotation guiding device can directly influence movement of the femoral component and the meniscal component relative to each other.

Furthermore, it may be of advantage for the rotation guiding device to be configured to enable sliding movement of the femoral component and the meniscal component relative to each other. In particular, the femoral component and the meniscal component can thus be formed congruent with each other, in order to achieve improved guidance of relative movement between femoral component and meniscal component.

In accordance with a further preferred embodiment of the invention, it may be provided that the rotation guiding device is configured to enable superimposed sliding/rolling movement of the femoral component and the meniscal component relative to each other. In this way, as a result of bending of the knee, for example, about a pivot axis extending transversely to the axis of rotation, translational movement of the femoral component relative to the meniscal component in the posterior direction is additionally enabled. Kinematics of a natural knee can thus be reproduced even better.

It is of advantage for the first and second guiding elements to be configured to define an angle of rotation of rotational movement of the meniscal component and the tibial component about the axis of rotation in dependence upon a flexion angle between femoral component and meniscal component. With the rotation guiding device it is thus possible to rotate the meniscal component relative to the tibial component about the axis of rotation in a defined manner, more particularly, in dependence upon a flexion or bending angle between femoral component and meniscal component. For example, the flexion angle can be defined by an angle between longitudinal axes of the tibia and the femur of the patient, on which the tibial component and the femoral component are anchored.

Preferably, the rotation guiding device is configured to enable exclusively rolling movement between the medial and/or lateral condylar surface and the medial and/or lateral joint surface. In this way, the knee joint can be stabilized particularly well, which is helpful, in particular, for patients with missing cruciate ligaments, in reducing risk of luxation.

The way in which the rotation guiding device functions can be easily improved by the first and second guiding elements comprising first and second guiding surfaces which bear at least partially on each other. Owing to the sliding or rolling of the first and second guiding element surfaces on each other, a desired movement between tibia and femur about the axis of rotation can thus be forced as a result of bending of the knee joint endoprosthesis.

Particularly good guidance is achieved by the second guiding element being formed in the posterior region of the femoral component. In particular, it can thus be influenced by a first guiding element which is arranged or formed, for example, in the anterior, i.e., in the front region of the tibial component. In particular, forces acting in the posterior direction can thus be introduced easily and safely from the tibial component into the femoral component during a bending movement.

The knee joint endoprosthesis can be constructed in a particularly simple and compact way by the second guiding element being formed on the femoral component in a region between the condyles. The functionality of the condyles is not limited thereby or only insignificantly. In particular, their size does not have to be reduced in comparison with conventional knee joint endoprostheses.

Expediently, the second guiding element surface comprises at least one convex surface region facing in the direction towards the tibial component. Such a surface region can interact in a desired manner, in particular, with a corresponding concave surface region of the first guiding element surface.

It is of advantage for the first guiding element surface to comprise at least one concave surface region facing in the direction towards the femoral component. Facing in the direction towards the femoral component means, in particular, that the concave surface region can be made to bear on a corresponding surface region of the femoral component, in particular, in order to force rotational movement of the femoral component relative to the tibial component about the axis of rotation as a result of bending of the knee.

The knee joint endoprosthesis can be constructed in a particularly simple way by the first and second guiding element surfaces taking the form of sliding surfaces. Here all kinds of sliding surfaces are possible.

To force defined rotational movement of the femoral component and the tibial component relative to each other, it is of advantage for the first and/or the second guiding element surfaces to be of unsymmetrical configuration in relation to a sagittal plane.

Forces in the posterior direction can be easily introduced from the first guiding element into the second guiding element if the first guiding element is formed in the anterior region of the tibial component.

The meniscal component can be mounted particularly easily and securely on the tibial component if the tibial component comprises a tibial surface facing in the direction towards the meniscal component.

The tibial component is particularly easy to produce if the tibial surface defines a tibial plane.

The construction of the knee joint endoprosthesis can be further simplified by the axis of rotation extending perpendicularly to the tibial plane.

For optimized sliding movement of the meniscal component and the tibial component relative to each other, it is expedient for the meniscal component to comprise an underside which has at least one plane surface region. It may, of course, also have two, three, four or more plane surface regions, which are separate from each other or else joined to each other.

Preferably, the meniscal component comprises two plane surface regions separate from each other.

It is expedient for each plane surface region to be associated with one of the two joint surfaces of the meniscal component. In particular, this can be achieved by the meniscal component comprising two meniscal component regions, with a respective meniscal component region comprising one of the two joint surfaces of the meniscal component.

It is of advantage for the meniscal component to comprise a medial meniscal component region and a lateral meniscal component region, and for the lateral meniscal component region and the medial meniscal component region to be connected to each other by a connection element. In particular, they may be rigidly connected to each other, whereby stability of the knee joint endoprosthesis is significantly increased.

Connection of the lateral and medial meniscal component regions is particularly simple if the connection element takes the form of a web. The web itself may have any cross-sectional shape. It is preferably of parallelepipedal shape and has rounded-off edges. It is, however, also conceivable for the web to be of circular or oval cross section, for example.

In accordance with a further preferred embodiment of the invention, it may be provided that the connection element has a connection element surface bearing at least partially on the tibial surface. The connection element surface may therefore also form part of an underside of the meniscal component, in particular, a plane surface region, which is connected to or adjacent to further surface regions of the meniscal component. In this way, a total surface of the meniscal component bearing on the tibial component can be maximized, whereby stability of the knee joint endoprosthesis can be significantly improved.

It is also conceivable to integrate the first guiding element into the tibial surface. The first guiding element is advantageously configured to project from the tibial surface. In particular, it may be releasably connectable or permanently firmly connected to the tibial component. In particular, it may be formed integrally with the tibial component.

In accordance with a further preferred embodiment of the invention, a securing device may be provided for securing the meniscal component to the tibial component in a connected position in which the meniscal component and the tibial component are mounted for rotation about the axis of rotation. The securing device therefore serves the purpose of preventing disengagement of the meniscal component and the tibial component from each other, in particular, when the two components assume the connected position in which they can be rotated in a defined manner about the axis of rotation as a result of bending of the knee.

A particularly simple construction of the securing device can be achieved by it comprising interacting first and second securing elements which are formed, on the one hand, on the meniscal component and, on the other hand, on the tibial component.

It is expedient for the first and second securing elements to comprise stop surfaces extending transversely to the axis of rotation to prevent movement of the meniscal component and the tibial component away from each other in the connected position. The stop surfaces prevent, in particular, movability of the meniscal component and the tibial component relative to each other in a direction parallel to the axis of rotation, more particularly, optionally towards each other and/or away from each other.

It is expedient for the first securing element to comprise a first retaining projection arranged or formed on the tibial component, and for the second securing element to comprise a second retaining projection arranged on the meniscal component. The first and second retaining projections may bear on each other, in particular, in the connected position. Furthermore, the retaining projections may also include or comprise the stop surfaces described above.

A particularly compact construction of the knee joint endoprosthesis can be achieved, in particular, by the first retaining projection being arranged or formed at or in a recess of the first guiding element. The recess may, for example, take the form of an indentation or a groove on the first guiding element. In particular, the recess may be partially delimited by the tibial surface of the tibial component.

The first guiding element preferably comprises the first retaining projection. A particularly compact construction of the knee joint endoprosthesis can thus be achieved.

Expediently, the first retaining projection comprises a first stop surface which is spaced from the tibial plane. For example, the connection element can thus be inserted fully or partly between the first stop surface and the tibial surface in order to prevent movement of the meniscal component and the tibial component relative to each other in a direction parallel to the axis of rotation.

The connection element preferably comprises or forms the second retaining projection. The connection element can thus interact, in particular, directly with the first retaining projection which, for example, is provided or formed on the first guiding element.

In particular, it is expedient for an upper side of the connection element to comprise or form a second stop surface of the securing device. In this way, the knee joint endoprosthesis or its securing device can be of particularly compact construction.

Preferably, the first guiding element surface is arranged or formed so as to be offset in the posterior direction in relation to the first stop surface. In this way, it can, in particular, be ensured that the functioning of the securing device and the rotation guiding device are spatially separate from each other.

In accordance with a preferred embodiment, it may be provided that the meniscal component and the tibial component can be brought from the connected position into an assembly position in which the first and second stop surfaces are in disengagement by rotation about the axis of rotation through a release angle. In particular, this can be achieved by the meniscal component and the tibial component being rotated relative to each other about the axis of rotation to such an extent that the stop surfaces of the securing device are no longer in engagement with each other or are no longer able to interact.

It is of advantage for the first and second stop surfaces to define in the connected position a surface section defined by at least partial overlapping of perpendicular projections thereof onto the tibial surface. In other words, the first and second stop surfaces can be brought into direct contact with each other in the connected position, but not in the assembly position.

It is also of advantage for the knee joint endoprosthesis to comprise a rotation delimiting stop for delimiting movement of the lateral joint surface in the anterior direction. In particular, the rotation delimiting stop may be configured to limit movement of the meniscal component relative to the tibial component about the axis of rotation so that movement of the lateral joint surface in the anterior direction is limited. The first guiding element preferably forms or comprises the rotation delimiting stop. The knee joint endoprosthesis can thus be constructed even more compactly.

It is of advantage for the femoral component and/or the meniscal component and/or the tibial component to be of integral construction. They may selectively or all be of integral construction.

In particular, it is of advantage for the femoral component and/or the tibial component to take the form of modular prosthesis parts. In particular, this means that the femoral component and/or the tibial component may respectively comprise a shaft which can be inserted into correspondingly prepared cavities on femur or tibia of the patient and fixed therein, for example, using screws or bone cement. The shafts may selectively be of one-part or multipart construction and may selectively be releasably connectable to or formed integrally, at least partly, with the femoral component or the tibial component. A modular design of femoral component and tibial component has the advantage that these can be individually adapted to the physiology of the patient.

The femoral component and the meniscal component are preferably made of different materials. The femoral component and also the tibial component are preferably made of an implant steel or some other bio-compatible metal, for example, titanium. The meniscal component is preferably made of an abrasion-resistant plastic material, for example, polyethylene or polyethylene with a high density and a high molecular weight.

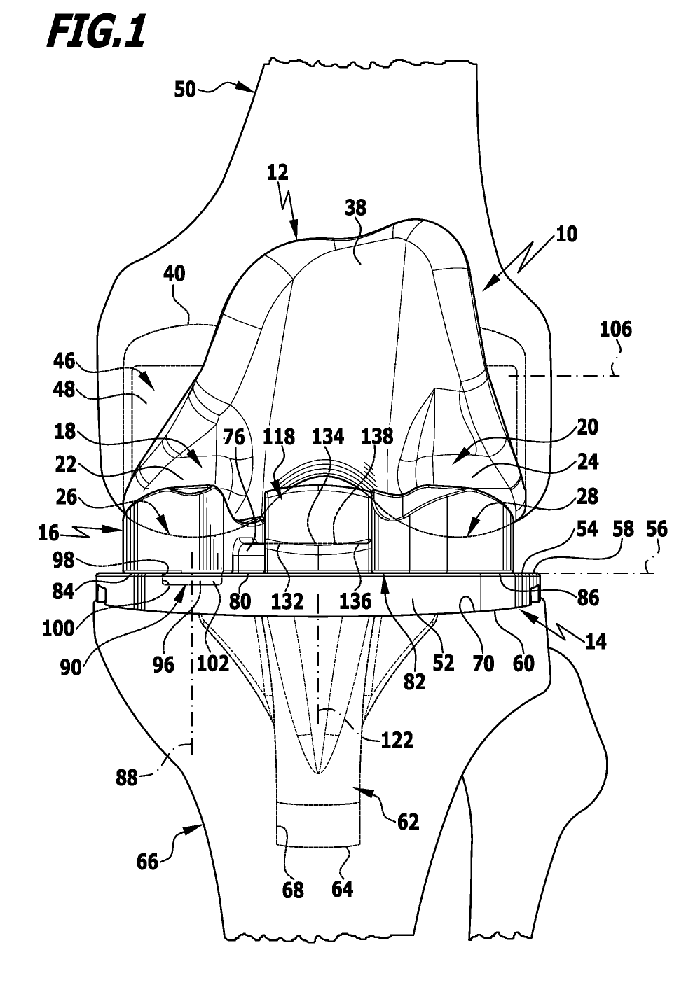

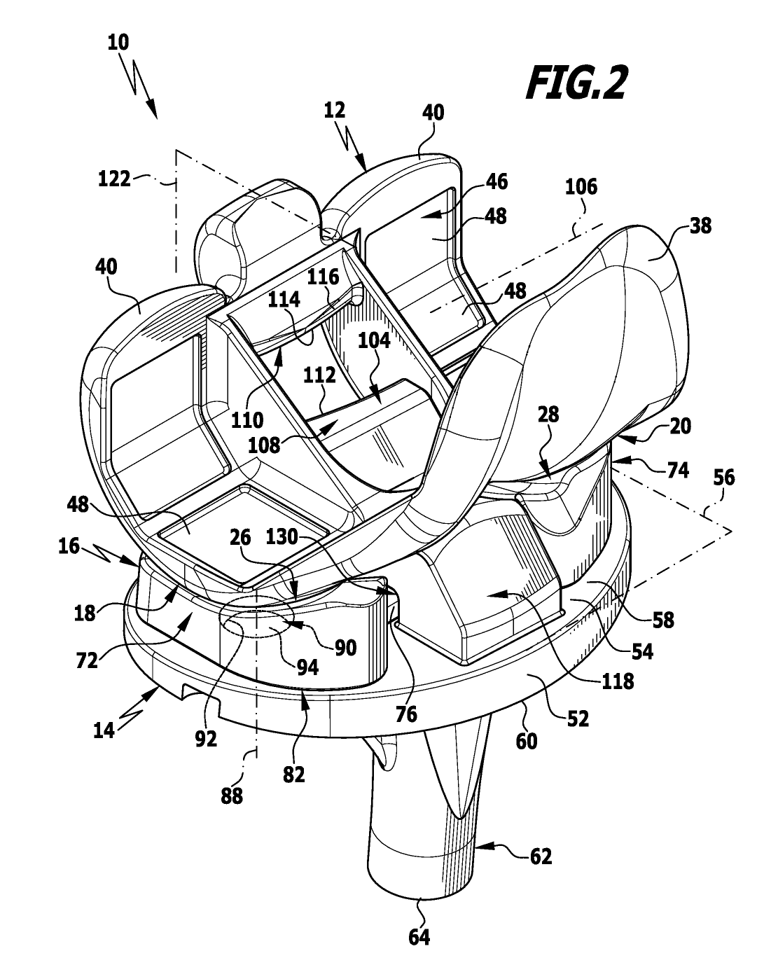

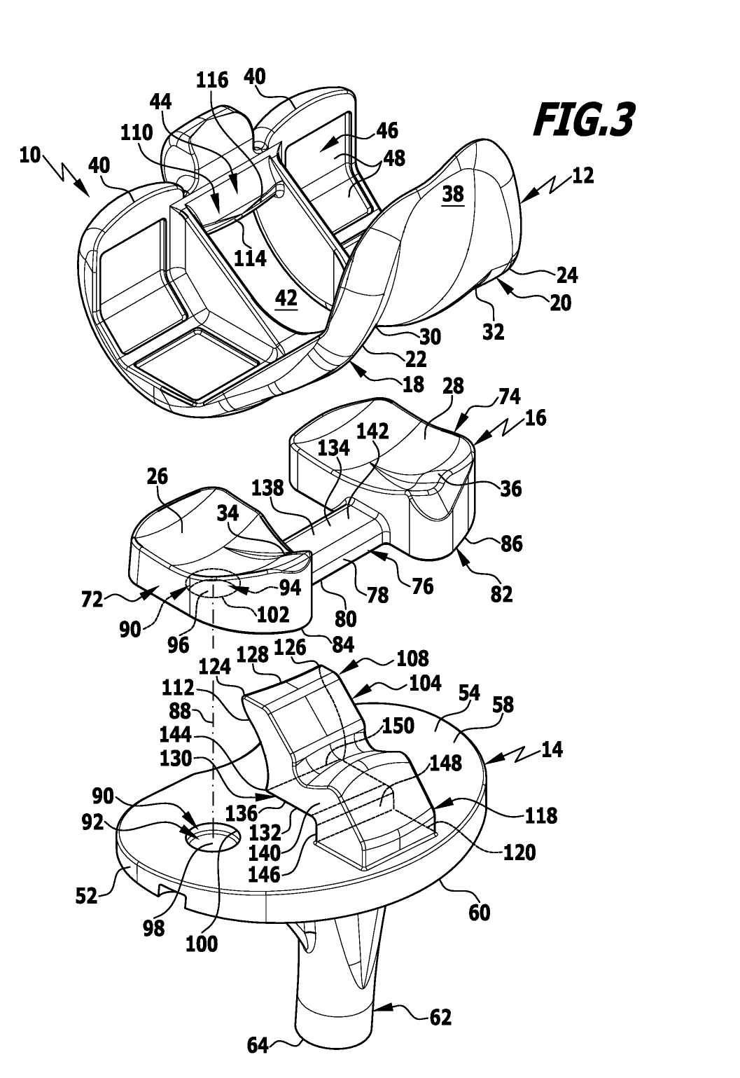

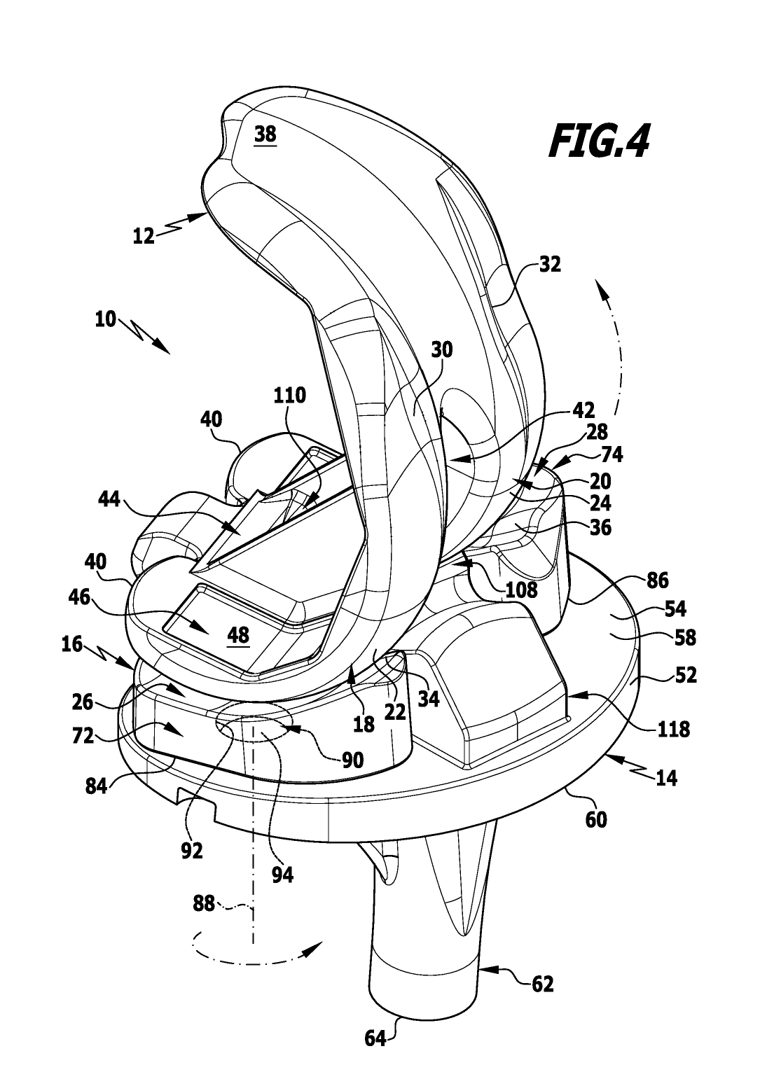

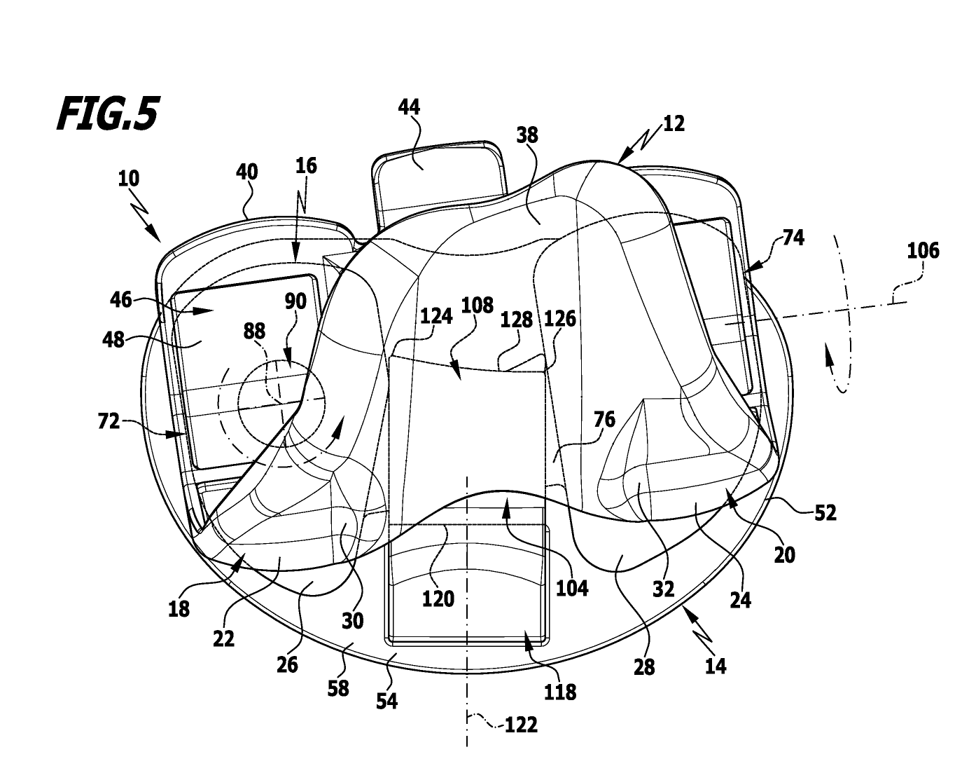

A first embodiment of a knee joint endoprosthesis, generally designated by reference numeral 10, which comprises a femoral component 12, a tibial component 14 and a meniscal component 16 movably mounted between the femoral component 12 and the tibial component 14, is shown diagrammatically in FIGS. 1 to 5.

The femoral component 12 comprises a medial condyle 18 and a lateral condyle 20, which have a medial condylar surface 22 and a lateral condylar surface 24, respectively. The medial and lateral condylar surfaces 22, 24 of the meniscal component 16 are substantially convexly curved, facing away from the femoral component 12, and are configured to slide or roll on medial and lateral joint surfaces 26, 28 of the meniscal component 16, on which they bear at least partially.

For improved guidance of relative movement of the femoral component 12 and the meniscal component 16 on each other, the medial and lateral condylar surfaces 22, 24 preferably each comprise a concavely curved condylar surface region 30, 32 substantially formed to correspond to interaction with convexly curved medial and lateral condylar surface regions 34, 36 of the otherwise substantially concavely curved medial and lateral joint surfaces 26, 28 facing away from the meniscal component 16. Radii of curvature of the medial and/or lateral joint surfaces 26, 28 are preferably configured larger than radii of curvature of the medial and/or lateral condylar surfaces 22, 24, so that not only a sliding movement is possible between the femoral component 12 and the meniscal component 16, but at the same time also a rolling movement, which, in particular, may be superimposed on the sliding movement.

The condyles 18 and 20 are connected to each other at their front or anterior end 38. Between their anterior end 38 and a posterior end 40 a gap or space 42 is formed between the two condyles 18 and 20 spaced from each other. In the region of their posterior ends 40, the condyles 18 and 20 are firmly connected to each other by a connection element 44.

The condylar surfaces 22 and 24 substantially form a front or outer side of the femoral component 12. Facing in the opposite direction, i.e., on a rear side or inner side of the condyles 18 and 20, bearing surfaces 46 are respectively formed which, in particular, may comprise a plurality of plane bearing surface regions 48 which are inclined relative to one another. To fix the femoral component 12 to a femur 50 of a patient, the femur 50 is prepared in accordance with the rear side of the femoral component 12, i.e., bearing surfaces, not shown in greater detail in the Figures, are prepared on the femur 50, which correspond to the bearing surfaces 46, in order to fix the femoral component 12, for example, with bone cement to the femur 50. Optionally, bone screws, not shown either, may be used to secure the femoral component 12 alternatively or additionally to the femur 50.

Also not shown in the Figures are alternative embodiments of femoral components 12, additionally comprising one or more shaft sections projecting from the femoral component 12, which can be inserted in corresponding recesses previously prepared on the femur 50 and fixed with bone cement and/or fastening elements such as, for example, bone screws. Such shafts or shaft sections may, in particular, be of modular construction, in order for their length to be matched in a correspondingly optimized manner to the patient's physiology.

The tibial component 14 comprises a plate 52 which has a tibial surface 54 facing in the direction towards the meniscal component 16 and defining a tibial plane 56. The tibial surface 54 is therefore plane. The tibial surface 54 forms an upper side 58 of the plate 52, which in a plan view is substantially kidney-shaped. A shaft or shaft section 62 is arranged or formed so as to project from an underside 60 of the plate 52. A distal end 64 thereof may optionally be connected to extension elements which can be inserted into a correspondingly prepared cavity 68 of a tibia 66. To fit the plate 52, the tibia 66 is partially resected, and a plane bearing surface 70 is prepared, on which the underside 58 bears substantially over a large area.

The meniscal component 16 comprises a medial meniscal component region 72 and a lateral meniscal component region 74. The lateral and medial meniscal component regions 72, 74 are of substantially parallelepipedal shape and are rigidly connected to each other by a web-shaped connection element 76. The medial joint surface 26 forms an upper side of the medial meniscal component region 72, the lateral joint surface 28 an upper side of the lateral meniscal component region 74. The connection element 76 which takes the form of a web 78 has a plane connection element surface 80 bearing on the tibial surface 54.

The meniscal component 16 comprises an underside 82, which has two plane surface regions 84 and 86, respectively, separate from each other. The surface regions 84 and 86 respectively form undersides of the meniscal component regions 72 and 74. The surface regions 84 and 86 are separated from each other by the connection element surface 80, but form with it the plane, continuous underside 82 of the meniscal component 16.

The meniscal component 16 is mounted for rotation at the medial side about an axis of rotation 88 relative to the tibial component 14. The axis of rotation 88 extends perpendicularly to the tibial plane 56. It is defined by a rotary bearing 90 formed between the tibial component 14 and the meniscal component 16. The rotary bearing 90 comprises interacting first and second bearing elements 92, 94, one being arranged or formed on the tibial component 14 and the other on the meniscal component 16. One of the bearing elements 92, 94 takes the form of a projection 96, the other bearing element the form of a corresponding recess 98. In the embodiment of the knee joint endoprosthesis 10 shown in the Figures, the projection 96 is formed so as to project from the surface region 84 in the form of a flat cylinder which engages substantially with positive locking the flat, hollow-cylindrical recess 98. The bearing elements 92, 94 are therefore formed rotationally symmetrically in relation to the axis of rotation 88. The projection 96 and the recess 98 have first and second guiding surfaces 100, 102, which are formed rotationally symmetrically in relation to the axis of rotation 88 and interact to guide a relative movement between tibial component 14 and meniscal component 16. The first guiding surface 100 is defined by a closed, ring-shaped wall surface of the recess 98, the second guiding surface 102 by a ring-shaped, closed outer surface of the cylindrical projection 96. The guiding surfaces 100 and 102 are therefore also formed coaxially in relation to the axis of rotation. The meniscal component 16 and the tibial component 14 are prevented from becoming displaced relative to each other parallel to the tibial plane by the described configuration of the projection and the recess. In the embodiment shown in the Figures, only rotation of the meniscal component 16 and the tibial component 14 relative to each other about the axis of rotation 88 is possible.

Alternatively, the rotary bearing 90 may also take the form of a ball joint bearing, not shown in the Figures, which comprises first and second ball joint surfaces, one of the ball joint surfaces being of hollow-spherical shape and the other ball joint surface of spherical shape. Preferably, one of the bearing elements 92, 94 then comprises the hollow-spherical ball joint surface and the other the spherical ball joint surface. For example, the hollow-spherical joint surface could be formed on the tibial component 14, but it is also conceivable to form the hollow-spherical ball joint surface on the meniscal component 16. To obtain optimum guidance of the bearing elements 92 and 94 on each other, the first and second ball joint surfaces in a ball joint bearing are formed so as to have identical or substantially identical radii of curvature.

The knee joint endoprosthesis 10 further comprises a rotation guiding device 104 for forcing rotational movement of the meniscal component 16 relative to the tibial component 14 about the axis of rotation 88 as a result of pivotal movement of the femoral component 12 and the tibial component 14 relative to each other about a pivot axis 106 extending transversely to the axis of rotation 88. The rotation guiding device 104 comprises interacting first and second guiding elements 108, 110 which are arranged or formed, on the one hand, on the tibial component 14 and, on the other hand, on the femoral component 12. The first and second guiding elements 108, 110 comprise first and second guiding element surfaces 112, 114 which, at least from a certain flexion angle between femur 50 and tibia 66 onwards, bear at least partially on each other.

The second guiding element 110 is formed in the posterior region of the femoral component 12. It is substantially formed by the connection element 44. The second guiding element surface 114 faces substantially in a direction into the space 42. It is otherwise formed on the femoral component 12 in the region between the condyles 18, 20. The second guiding element surface has at least one convex surface region 116 facing in the direction towards the tibial component 14.

The first guiding element 108 is formed in the anterior region of the tibial component 14. It takes the form of a projection 118 projecting from the tibial surface 54 and defining a rectangular surface region 120 on the tibial surface 54. The first guiding element surface 112 is spaced from the tibial surface 54 and concavely curved. In the lower region, in particular, in the region of the surface region 120, the first guiding element 108 is formed mirror-symmetrically in relation to a plane of symmetry 122 preferably defining a sagittal plane and simultaneously defining a plane of symmetry of the plate 52. The plane of symmetry 122 is oriented perpendicularly to the tibial plane 56. In the posterior region, i.e., in the region of the first guiding element surface 112, the first guiding element 108 is, however, formed unsymmetrically in relation to the plane of symmetry 122. A medial end 124 of the projection 118 projects further in the posterior direction than a lateral end 126. The first guiding element surface is therefore also formed unsymmetrically in relation to the plane of symmetry 122.

The first and second guiding element surfaces 112 and 114 take the form of slide surfaces. The first guiding element surface 112 also comprises a concave surface region 128 facing in the direction towards the femoral component 12 and bearing partially on the second guiding element surface 114 at least when the first and second guiding elements 108 and 110 are in contact and interact.

Owing to the special construction of the first and second guiding elements 108 and 110, the rotation guiding device 104 is configured overall to force rotation of the femoral component 12 relative to the tibial component 14 about the axis of rotation 88 when the femoral component 12 is pivoted about the pivot axis 106 relative to the meniscal component 116, i.e., when femur 50 and tibia 66 are brought from a stretched or extension position in which their longitudinal axes are aligned substantially parallel to each other into a flexion position. This is referred to as a flexion movement. The rotation guiding device 104 can, in particular, be configured to force rolling movement of the femoral component 12 and the meniscal component 16 on each other. In particular, this can be achieved by a curvature of the medial and/or lateral joint surfaces 26, 28 being larger than a curvature of the medial and lateral condylar surfaces 22, 24. If the radii of curvature of these surfaces are adapted to each other, then the rotation guiding device 104 can also be configured to enable sliding movement, or even exclusively such a sliding movement, of the femoral component and the meniscal component 16 relative to each other. Furthermore, the rotation guiding device 104 can also be configured to enable superimposed sliding/rolling movement of the femoral component 12 and the meniscal component 16 relative to each other. For example, this can be achieved by correspondingly provided radii of curvature of the condylar surfaces 42, 24 and the joint surfaces 26, 28, which are not identical.

Owing to their specially shaped first and second guiding element surfaces 112, 114, the first and second guiding elements 108, 110 are configured to define a rotational angle of a rotational or rotary movement of the meniscal component 16 and the tibial component 14 relative to each other about the axis of rotation 88 in dependence upon a flexion angle between femoral component 12 and tibial component 14. In other words, this means that the meniscal component 16 with its lateral meniscal component region 74 is rotated all the further in the posterior direction, the greater a bending or flexion angle is between femoral component 12 and tibial component 14, the bending angle being measurable, for example, starting from a stretched knee joint. Owing to the spacing between the first and second guiding element surfaces 112 and 114 in the embodiment shown in the Figures in the stretched or extension position, a forced rotation about the axis of rotation 88 only starts from a certain or minimal bending angle onwards. The minimal or required bending angle preferably ranges between 30° and 60°.

A securing device generally designated by reference numeral 130 is provided for securing the meniscal component 16 on the tibial component 14 in a connected position in which the meniscal component 16 and the tibial component 14 are mounted for rotation about the axis of rotation 88 and cannot be separated from each other. It comprises interacting first and second securing elements 132 and 134 which are formed, on the one hand, on the meniscal component 16 and, on the other hand, on the tibial component 14. The first and second securing elements 132 and 134 comprise stop surfaces 136 and 138 extending transversely to the axis of rotation 88 to prevent movement of the meniscal component 16 and the tibial component 14 in the connected position away from each other. The first securing element 132 comprises a first retaining projection 140 arranged or formed on the tibial component 14. The second securing element 134 comprises a second retaining projection 142 arranged on the meniscal component 16. The first retaining projection 140 is formed adjacent to a recess 144 of the first guiding element 108. All in all, the guiding element 108 comprises the first retaining projection 140. This comprises the first stop surface 136, which is spaced from the tibial surface 154 or the tibial plane 56.

The connection element 76 forms the second retaining projection 142. An upper side of the connection element 76 forms the second stop surface 138 of the securing device 130.

The recess 144 on the first guiding element 108 is of such dimensions that the connection element 76 can engage the recess 144. A side surface 146, facing in the posterior direction, of the first guiding element 108, which delimits the recess 144, forms a rotation delimiting stop 148 for delimiting movement of the lateral meniscal component region 74 in the anterior direction. In the extension position, the connection element 76 then preferably extends perpendicularly to the plane of symmetry 122 and in this position is preferably formed and aligned mirror-symmetrically in relation to the plane of symmetry 122. When the connection element 76 enters or engages at least partially the recess 144, the meniscal component 16 and the tibial component 14 assume the connected position described above. They can then not be moved relative to each other in a direction parallel to the axis of rotation 88. The meniscal component 16 and the tibial component 14 can, however, be transferred from the connected position to an assembly position in which the first and second stop surfaces 136 and 138 are in disengagement. This is achieved by rotating the meniscal component 16 relative to the tibial component 14 about the axis of rotation 88 through a release angle. In the connected position, perpendicular projections of the stop surfaces 136, 138 onto the tibial surface 54 overlap one another and thus define a projected surface section on the tibial surface 54. So long as the surface content of this surface section is greater than zero, the meniscal component 16 and the tibial component 14 assume the connected position. In the connected position, they are, however, freely movable relative to each other about the axis of rotation 88.

It should also be noted that the first guiding element surface 112 is offset in the posterior direction in relation to the first stop surface 136. In the first guiding element 108 shown in the Figures, the first guiding element surface 112 and the first stop surface 136 form a common edge 150.

Both the femoral component 12 and the tibial component 14 are preferably of integral construction. In the described embodiment, the meniscal component 16 is preferably also of integral construction. In particular, the femoral component 12 and the tibial component 14 may also optionally take the form of modular prosthesis parts. As already described above, in alternative embodiments, both the femoral component 12 and the meniscal component 16 can be equipped with modular shafts which can be adapted in length and diameter to the respective physiology of the patient.

The femoral component 12 and the tibial component 14 are preferably made of an instrument steel, the meniscal component 16 of a highly abrasion-resistant plastic material.

1. A knee joint endoprosthesis comprising:
a femoral component, a tibial component and a meniscal component mounted between the femoral component and the tibial component, said meniscal component being mounted on the tibial component for rotation about an axis of rotation extending at the medial side, wherein a rotation guiding device is provided to force rotational movement of the meniscal component relative to the tibial component about the axis of rotation as a result of pivotal movement of the femoral component and the tibial component relative to each other about a pivot axis extending transversely to the axis of rotation, said rotation guiding device having interacting first and second guiding elements arranged or formed, on the one hand, on the femoral component and, on the other hand, on the tibial component.

2. The knee joint endoprosthesis in accordance with claim 1, wherein the femoral component comprises a medial condyle and a lateral condyle, which have a medial condylar surface and a lateral condylar surface, and the meniscal component comprises a medial joint surface and a lateral joint surface on which the medial and lateral condylar surfaces bear at least partially.

3. The knee joint endoprosthesis in accordance with claim 2, wherein the medial and/or the lateral condylar surface comprises a concavely curved condylar surface region, and wherein the medial and/or the lateral joint surface comprises a convexly curved joint surface region corresponding to the medial and/or the lateral condylar surface.

4. The knee joint endoprosthesis in accordance with claim 2, wherein radii of curvature of the medial and/or lateral joint surfaces are larger than radii of curvature of the medial and/or lateral condylar surfaces.

5. The knee joint endoprosthesis in accordance with claim 1, wherein the axis of rotation is defined by a rotary bearing formed between the tibial component and the meniscal component.

6. The knee joint endoprosthesis in accordance with claim 5, wherein the rotary bearing comprises interacting first and second bearing elements which are arranged or formed, on the one hand, on the tibial component and, on the other hand, on the meniscal component.

7. The knee joint endoprosthesis in accordance with claim 6, wherein one of the bearing elements is of cylindrical shape and the other bearing element is of hollow-cylindrical shape.

8. The knee joint endoprosthesis in accordance with claim 1, wherein the rotation guiding device is configured to force rolling movement of the femoral component and the meniscal component on each other.

9. The knee joint endoprosthesis in accordance with claim 1, wherein the first and second guiding elements are configured to define an angle of rotation of rotational movement of the meniscal component and the tibial component about the axis of rotation in dependence upon a flexion angle between femoral component and tibial component.

10. The knee joint endoprosthesis in accordance with claim 1, wherein the first guiding element is formed in the anterior region of the tibial component.

11. The knee joint endoprosthesis in accordance with claim 1, wherein the tibial component comprises a tibial surface facing in the direction towards the meniscal component.

12. The knee joint endoprosthesis in accordance with claim 1, wherein the meniscal component comprises an underside which has at least one plane surface region.

13. The knee joint endoprosthesis in accordance with claim 12, wherein the meniscal component comprises two plane surface regions separate from each other.

14. The knee joint endoprosthesis in accordance with claim 13, wherein each plane surface region is associated with one of the two joint surfaces of the meniscal component.

15. The knee joint endoprosthesis in accordance with claim 1, wherein the meniscal component comprises a medial meniscal component region and a lateral meniscal component region, and wherein the lateral meniscal component region and the medial meniscal component region are connected to each other by a connection element.

16. The knee joint endoprosthesis in accordance with claim 15, wherein the connection element takes the form of a web.

17. The knee joint endoprosthesis in accordance with claim 11, wherein the first guiding element is configured to project from the tibial surface.

18. The knee joint endoprosthesis in accordance with claim 1, characterized by a securing device for securing the meniscal component to the tibial component in a connected position in which the meniscal component and the tibial component are mounted for rotation about the axis of rotation.

19. The knee joint endoprosthesis in accordance with claim 18, wherein the securing device comprises interacting first and second securing elements which are formed, on the one hand, on the meniscal component and, on the other hand, on the tibial component.

20. The knee joint endoprosthesis in accordance with claim 19, wherein the first and second securing elements comprise stop surfaces extending transversely to the axis of rotation to prevent movement of the meniscal component and the tibial component away from each other in the connected position.

21. The knee joint endoprosthesis in accordance with claim 19, wherein the first securing element comprises a first retaining projection arranged or formed on the tibial component, and wherein the second securing element comprises a second retaining projection arranged on the meniscal component.

22. The knee joint endoprosthesis in accordance with claim 20, wherein an upper side of the connection element comprises or forms a second stop surface of the securing device.

23. The knee joint endoprosthesis in accordance with claim 1, characterized by a rotation delimiting stop for delimiting movement of the lateral joint surface in the anterior direction.

24. The knee joint endoprosthesis in accordance with claim 23, wherein the first guiding element forms or comprises the rotation delimiting stop.

25. The knee joint endoprosthesis in accordance with claim 1, wherein the femoral component and/or the tibial component take the form of modular prosthesis parts.
